(19) United States
(12) Patent Application Publication
(10) Pub. No.: US 2025/0215106 A1
(43) Pub. Date: Jul. 3, 2025

(54) COMPOSITIONS AND METHODS FOR TREATING IDIOPATHIC NEPHROTIC SYNDROME

(71) Applicant: THE REGENTS OF THE UNIVERSITY OF COLORADO, A BODY CORPORATE, Denver, CO (US)

(72) Inventors: Gabriel CARA-FUENTES, Denver, CO (US); Richard JOHNSON, Denver, CO (US)

(21) Appl. No.: 18/858,888

(22) PCT Filed: Apr. 19, 2023
(86) PCT No.: PCT/US2023/065969

§ 371 Date: Oct. 22, 2024

Related U.S. Application Data
(60) Provisional application No. 63/363,463, filed on Apr. 22, 2022.

(51) Int. Cl. C07K 16/28

(52) U.S. Cl. CPC C07K 16/2896; C07K 2317/76

(57) ABSTRACT

Methods of treating idiopathic nephrotic syndrome in a subject involve reducing urinary and/or blood levels of CD93, reducing or inhibiting CD93-mediated podocyte activation. or reducing or inhibiting CD93 release from glomerular endothelial cells. The methods may include administration of a composition including at least one of an anti-CD93 antibody and an inhibitor of pi integrin.

### CROSS REFERENCE TO RELATED APPLICATION

The present application claims the benefit of priority under 35 U.S.C. § 119 to U.S. Provisional Patent Application No. 63/363,463, entitled “NOVEL THERAPEUTIC TARGET FOR NEPHROTIC SYNDROME,” filed Apr. 22, 2022, the entirety of which is hereby incorporated by reference herein for all purposes.

### TECHNICAL FIELD

The present disclosure relates generally to compositions and methods for treating idiopathic nephrotic syndrome.

### BACKGROUND

Idiopathic nephrotic syndrome (INS) refers to a type of nephrotic syndrome with a heterogeneous clinical course that associates with a spectrum of histological patterns, including minimal change disease (MCD) and focal segmental glomerulosclerosis (FSGS). It is considered strictly a podocyte disorder triggered by circulating factors but the mechanisms of podocyte injury are unknown. INS is characterized by episodes of severe proteinuria and hypoalbuminemia often associated with dyslipidemia and edema. Loss of serum proteins leads to a hypercoagulable state, a higher rate of infectious diseases, and fluid balance dysregulation. It affects two to ten children per 100,000 per year. with a cumulative prevalence of 16 per 100,000 children. making it the most frequent glomerular disease in childhood. INS accounts for 15-30% of adult glomerulopathies. Accordingly, compositions and methods for treating INS are needed.

### SUMMARY

Embodiments disclosed herein relate to compositions and methods for treating idiopathic nephrotic syndrome by reducing urinary and/or blood levels of CD93, reducing the effects of CD93 on podocytes, or reducing or inhibiting CD93 release from glomerular endothelial cells.

In accordance with embodiments of the present disclosure, a method of treating idiopathic nephrotic syndrome may involve reducing urinary and/or blood levels of CD93. In some examples, the idiopathic nephrotic syndrome is at least one of minimal change disease, focal segmental glomerulosclerosis, steroid sensitive nephrotic syndrome, steroid resistant nephrotic syndrome, frequent relapsing nephrotic syndrome, or infrequent relapsing nephrotic syndrome. In some examples, urinary and/or blood levels of CD93 are reduced by administering at least one anti-CD93 antibody. In some examples, urinary and/or blood levels of CD93 are reduced by reducing or inhibiting CD93 release from glomerular endothelial cells. In some examples, urinary and/or blood levels of CD93 are reduced by administering a composition capable of reducing or inhibiting CD93 release from glomerular endothelial cells. In some implementations, the method further includes preventing or reducing podocyte activation. In some examples, podocyte activation includes phosphorylation of focal adhesion kinase and reduction of activation includes administration of an inhibitor of β1 integrin, which may include monoclonal antibody 13. In some examples, podocyte activation includes phosphorylation of focal adhesion kinase and reduction of activation includes administration of at least one anti-CD93 antibody.

In accordance with embodiments of the present disclosure, a method of preventing or reducing podocyte injury may involve reducing or inhibiting CD93-mediated podocyte activation. In some examples, podocyte activation includes phosphory lation of FAK and reduction of activation includes administration of an inhibitor of β1 integrin, which may include monoclonal antibody 13. In some examples, podocyte activation is reduced by administering at least one anti-CD93 antibody. In some examples, podocyte activation is reduced by reducing or inhibiting CD93 release from glomerular endothelial cells. In some examples, podocyte activation is reduced by administering a composition capable of reducing or inhibiting CD93 release from glomerular endothelial cells.

In accordance with embodiments of the present disclosure, a method of treating an idiopathic nephrotic syndrome may involve reducing or inhibiting CD93 protein release from glomerular endothelial cells. In some examples, the idiopathic nephrotic syndrome is at least one of minimal change disease, focal segmental glomerulosclerosis. steroid sensitive nephrotic syndrome, steroid resistant nephrotic syndrome, frequent relapsing nephrotic syndrome, or infrequent relapsing nephrotic syndrome. In some examples, the method includes administering a composition capable of reducing or inhibiting CD93 release. In some implementations, CD93 levels are reduced in urine and/or blood. In some implementations, podocyte activation is reduced.

In accordance with embodiments of the present disclosure, a method of diagnosing idiopathic nephrotic syndrome relapse and/or progression to end stage kidney disease may involve measuring levels of CD93 protein in urine. In examples, a high level of urinary CD93 indicates likelihood of kidney disease progression. In examples. a high level of urinary CD93 indicates a higher risk of future proteinuria. In examples, a low level of urinary CD93 indicates a shorter time to complete remission. In examples, urinary CD93 levels of about 750 ng/g creatinine or greater are considered high. In examples, urinary CD93 levels of less than 750 ng/g creatinine are considered low.

This Summary is neither intended to be, nor should it be, construed as being representative of the full extent and scope of the present disclosure. Moreover, references made herein to “the present disclosure,” or aspects thereof, should be understood to mean certain embodiments of the present disclosure and should not be construed as limiting all embodiments to a particular description. The present disclosure is set forth in various levels of detail in this Summary as well as in the attached drawings and Detailed Description and no limitation as to the scope of the present disclosure is intended by either the inclusion or non-inclusion of elements, components, etc. in this Summary. Features from any of the disclosed embodiments may be used in combination with one another without limitation. In addition, other features and advantages of the present disclosure will become apparent to those of ordinary skill in the art through consideration of the following Detailed Description and the accompanying drawings.

### BRIEF DESCRIPTION OF THE DRAWINGS

FIG. 1A shows immunofluorescence images of control, MCD, or FSGS juvenile and adult human kidney tissue probed for CD93 or lectin ulex europaeus agglutinin I. FIG. 1B is a densitometric analysis of FIG. 1A.

FIGS. 2A and 2B show CD93 levels in urine (FIG. 2A) and sera (FIG. 2B) of patients with MCD/INS in relapse or remission, or control subjects.

FIG. 3 shows CD93 levels in supernatants from GEnC cells previously cultured with sera from pediatric patients with INS in relapse or from control subjects.

FIG. 4A is a Western blot and FIG. 4B is a densitometric analysis of the same. each showing a CD93 time-dependent increase in pFAK, which was abrogated by pre-treatment with the β1 integrin inhibitor mAb 13.

FIG. 5A is a Western blot and FIG. 5B is a densitometric analysis of the same. each showing the effects of MCD in relapse sera (+/−anti-CD93 antibody) on FAK phosphorylation in cultured human podocytes.

FIG. 6 is a graph showing the CD93 mRNA levels from microdissected human glomeruli of control or INS (MCD or FSGS) subjects, the latter separated by their proteinuria levels.

FIGS. 7A and 7B are graphs showing the CD93 levels from control or INS subjects, the latter separated by their proteinuria levels.

FIGS. 8A-8C are Kaplan-Meier curves showing the relationship between urinary CD93 and several clinic outcomes.

FIG. 9A is a Western blot and FIG. 9B is a densitometric analysis of the same. each showing CD93 protein expression in GEnC cell lysates cultured with sera from patients with INS in relapse or from healthy control subjects

FIG. 10 is a Western blot of the co-immunoprecipitation of CD93 and β1 integrin in cultured human podocytes.

FIG. 11A is a bar graph showing CD93 levels in urine from a PAN rat model of proteinuria compared to normal saline controls. FIG. 11B shows immunofluorescence images of control or PAN kidney tissue probed for CD93.

### DETAILED DESCRIPTION

This disclosure relates to compositions and methods for treating or diagnosing idiopathic nephrotic syndrome (INS). The treatment methods disclosed herein involve reducing urinary and/or blood levels of CD93, reducing or inhibiting CD93-mediated podocyte activation, and reducing or inhibiting CD93 release from glomerular endothelial cells (GEnC). In some implementations, the methods involve administration of a composition capable of reducing urinary and/or blood levels of CD93, reducing or inhibiting CD93-mediated podocyte activation, and/or reducing or inhibiting CD93 release from GEnC. The compositions may include at least of an anti-CD93 antibody and an inhibitor of β1 integrin.

GEnC are endothelial cells that line the inner aspect of the glomerular capillary. CD93 (cluster of differentiation 93) is a transmembrane protein predominantly expressed by the endothelium. In inflammatory states, the extracellular domain of CD93 can be cleaved or shed from the cell surface to produce a soluble form of the protein. In the endothelium, CD93 maintains the endothelial barrier function and regulates cell migration via its ability to activate the β1 integrin-focal adhesion kinase (FAK) signaling pathway. FAK is a cy toplasmic tyrosine kinase and mediator of intracellular signaling by integrins. Upon activation by integrins through disruption of an auto-inhibitory mechanism. FAK undergoes autophosphorylation. FAK activation in podocytes leads to proteinuria in experimental models and has been reported in minimal change disease, a cause or type of INS.

As used herein, “idiopathic nephrotic syndrome” or “INS” refers to any nephrotic syndrome that occurs in the absence of an identifiable systemic cause. A biopsy may or may not be performed to identify a subtype or classification of INS, such as minimal change disease (MCD), focal segmental glomerulosclerosis (FSGS), steroid sensitive nephrotic syndrome (SSNS), steroid resistant nephrotic syndrome (SRNS). frequent relapsing nephrotic syndrome (FRNS), or infrequent relapsing nephrotic syndrome (IRNS).

Treating INS, as contemplated herein, encompasses treating, preventing, delaying, or reversing at least one symptom of INS. Accordingly, “treating” or “treatment” or “alleviation” refers to both therapeutic treatment and prophylactic or preventative measures, wherein the object is to prevent, postpone, or slow down (lessen) the targeted pathological condition, disorder and/or symptom. Treatment may lead to remission, which may be temporary until a relapse, or may be permanent. Treatment may include preventing a relapse. Those in need of treatment include those already with the condition as well as those prone to have the condition or those in whom the condition is to be prevented. A subject is successfully “treated” for INS if, after receiving a therapeutic amount of a composition according to methods of this disclosure, the subject shows observable and/or measurable reduction in. or absence of, a symptom such as proteinuria. Proteinuria may be measured by urinary protein/creatinine ratio (UPCR) or urine dipstick. A UPCR of about 0.3 to about 2 g/g may be considered indicative of an active disease state: UPCR >about 2 g/g or ≥3 by urine dipstick may be considered indicative of relapse: and UPCR <0.3 g/g or negative/trace urine dipstick may be considered indicate of remission. The terms “treat” or “treating” are used consistently herein for ease of illustration, only, and thus should not be construed as limiting.

An “effective amount” of a composition is an amount sufficient to carry out a specifically stated purpose. An “effective amount” may be determined empirically and in a routine manner, in relation to the stated purpose. The term “therapeutically effective amount” refers to an amount of a composition to “treat” INS in a subject.

As used herein. “subject” means a human or other mammal. Non-human subjects may include, but are not limited to, various mammals including domestic pets and/or livestock. A subject can be considered in need of treatment. The disclosed methods and systems may be effective to treat healthy human subjects, patients diagnosed with INS, or patients experiencing proteinuria.

“Reducing,” “reduce,” or “reduction” means decreasing the severity, scope. or degree of INS or a symptom thereof or a cause thereof. For example, “reducing” urinary and/or blood levels of CD93 means decreasing the relatively higher amounts of urinary or circulating levels of CD93, respectively, that may characterize a patient with INS, the decrease being towards or to levels that characterize a non-disease state.

“Administration of” and “administering a” compound, composition, or agent should be understood to mean providing a compound. composition. or agent: a prodrug of a compound. composition, or agent: or a pharmaceutical composition as described herein. The compound. composition, or agent can be provided or administered by another person to the subject (e.g., intravenously, subcutaneously, intramuscularly) or it can be self-administered by the subject (e.g., orally or by inhalation). The compound, composition, or agent may be an anti-CD93 antibody or an anti-β1 integrin antibody.

As used herein. the term “antibody” refers to an immunoglobulin (Ig) whether natural or partly or wholly synthetically produced. The term also covers any polypeptide or protein having a binding domain which is. or is homologous to, an antigen-binding domain. The term further includes “antigen-binding fragments.”

“Pharmaceutical compositions” or “pharmaceutical formulations” are compositions that include an amount (for example, a unit dosage) of one or more of the disclosed active ingredients together with one or more non-toxic pharmaceutically acceptable additives, including carriers, diluents. and/or adjuvants. and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19^{th} Edition).

As used herein, a “pharmaceutically acceptable excipient” or a “pharmaceutically acceptable carrier” means a pharmaceutically acceptable material. composition, or vehicle involved in giving form or consistency to the pharmaceutical composition. Each excipient or carrier should be compatible with other ingredients of the pharmaceutical composition when comingled such that interactions that would substantially reduce the efficacy of the formulations of this disclosure when administered to a subject and interactions that would result in pharmaceutical compositions that are not pharmaceutically acceptable are avoided. In addition, each excipient or carrier should be of sufficiently high purity to render it pharmaceutically acceptable.

The singular terms “a,” “an,” and “the” include plural referents unless context clearly indicates otherwise. Similarly, the word “or” is intended to include “and” unless the context clearly indicates otherwise. The term “comprises” means “includes.” Also, “comprising A or B” means including A or B, or A and B, unless the context clearly indicates otherwise. The term “about” intended to include values or amounts up to and including 10% greater than or less than the recited value or amount. Although methods and materials similar or equivalent to those described herein can be used in the practice or testing of this disclosure. suitable methods and materials are described below. In addition, the materials, methods, and examples are illustrative only and not intended to be limiting.

Unless otherwise defined. all technical and scientific terms used herein have the same meaning as commonly understood by one of ordinary skill in the art to which this disclosure belongs. In the case of conflict, the present specification, including definitions, will control. All publications, patent applications, patents, and other references mentioned herein are incorporated by reference. The references cited herein are not admitted to be prior art.

### Therapeutic Methods

The methods described herein are suitable for treating or preventing INS or at least one symptom or cause thereof. The INS may include or be caused by minimal change disease (MCD), focal segmental glomerulosclerosis (FSGS), steroid sensitive nephrotic syndrome (SSNS), steroid resistant nephrotic syndrome (SRNS), frequent relapsing nephrotic syndrome (FRNS), or infrequent relapsing nephrotic syndrome (IRNS). Symptoms may include proteinuria, hypoalbuminemia, dyslipidemia, and/or edema.

Patients experiencing INS may have elevated levels of CD93 in their urine and/or blood. such as in the serum, compared to healthy subjects. The disclosed methods may reduce urinary and/or blood levels of CD93. In some implementations, urinary and/or blood levels of CD93 are reduced by reducing or inhibiting CD93 release from GEnC, as described below. In some implementations, urinary and/or blood levels of CD93 are reduced by administering a composition capable of reducing urinary and/or blood levels of CD93. The composition may bind, sequester, cleave, disable, eliminate, or otherwise block the activity of CD93. For example, the composition may prevent CD93 from interacting with a kinase target, such as FAK. In other examples, the composition may prevent CD93 from interacting with an integrin target. such as β1 integrin. The composition may include an anti-CD93 antibody. The antibody may be specific to the extracellular domain of CD93. An example of a suitable antibody is HPA009300 from Sigma Aldrich.

Patients experiencing INS may have more activated podocytes, or podocytes with higher activation levels, compared to healthy subjects. The activation may be caused. directly or indirectly, by CD93. Without being limited to any mechanism or mode of action, CD93 may activate the β1 integrin-FAK signaling pathway, which in turn may activate podocytes. Consistent with this theory, culturing human podocytes with recombinant CD93 leads to an increase in pFAK expression, and that expression is abrogated when podocytes are preemptively cultured with the β1 integrin inhibitor, mAb 13. See Example 4. Sera from patients with INS in relapse cause FAK activation in podocytes, and the activation is mitigated when podocytes are exposed to the same INS sera mixed with an anti-CD93 antibody. See Example 5.

The disclosed methods may reduce or inhibit podocyte activation, such as CD93-dependent podocyte activation. In some implementations. podocyte activation is reduced or inhibited by reducing or inhibiting CD93 release from GEnC, as described below: In some implementations. podocyte activation is reduced or inhibited by administering a composition capable of reducing or inhibiting podocyte activation. The composition may bind. sequester. cleave. disable, eliminate. or otherwise block the activity of CD93. For example, the composition may prevent CD93 from interacting with a kinase target, such as FAK. In other examples, the composition may prevent CD93 from interacting with an integrin target, such as β1 integrin. The composition may include an anti-CD93 antibody. The antibody may be specific to the extracellular domain of CD93. An example of a suitable antibody is HPA009300 from Sigma Aldrich. Additionally or alternatively. the composition may block at least a portion of the β1 integrin-FAK signaling pathway. The composition may include an anti-β1 integrin antibody. An example of a suitable antibody is mAb 13.

In patients experiencing INS, a portion of the transmembrane protein CD93, such as the extracellular portion. may be cleaved or otherwise shed from GEnC. The amount of CD93 released may be higher in INS patients compared to healthy subjects. The disclosed methods may reduce or inhibit CD93 release from GEnC. In some implementations. CD93 release from GEnC is reduced or inhibited by administering a composition capable of reducing or inhibiting GEnC release. Reducing or inhibiting CD93 release from GEnC may reduce urinary and/or blood levels of CD93 and/or may reduce podocyte activation.

The compositions of this disclosure may be administered to a subject before or after onset of INS. The compositions may be administered after onset of INS to treat INS. The compositions may be administered when a patient is in remission from INS to prevent or reduce the likelihood of a relapse. The frequency and duration of composition administration may vary. For example, an effective amount of a composition may be administered once a day for one week to treat INS. Doses may be administered more than once a day, such as two to six times per day. Doses may be administered for longer than one week, such as two, three, four, five, six. or more weeks. In one example, an oral or inhalable composition is administered once or twice a day for about eight weeks. In another example, an injectable composition is administered by intravenous injection once a week for about eight weeks.

The number of times per day, week, or month that the disclosed compositions are administered to a subject. along with the entire duration of the treatment period. may depend on the severity or type of condition a subject is experiencing or is expected to experience. For example, embodiments in which a composition is administered to treat existing INS may involve more frequent administrations than embodiments in which a composition is administered to prevent a relapse or reduce the severity of a relapse. Embodiments in which composition is administered to prevent a relapse or reduce the severity of a relapse may involve a longer treatment period than embodiments in which a composition is administered to treat existing INS. The length of the treatment period may also be patient-specific and re-evaluated periodically by a nephrologist or other health care provider.

### Pharmaceutical Formulations

Compositions of this disclosure may include active ingredients such as an antibody against CD93 or against β1 integrin and may be administered as pharmaceutical formulations. Active ingredients of this disclosure may be formulated into a pharmaceutical dosage form adapted for inhalation or intravenous, intra-arterial, subcutaneous, or intraperitoneal injection may also be used. Injection by such routes may use an injection device, such as an IV drip device, infusion pump, and/or tuberculin syringe.

In embodiments, the active ingredient may be administered concurrently with one or more excipients. Suitable excipients may vary depending upon the particular dosage form chosen. In addition, suitable pharmaceutically acceptable excipients may be chosen for a particular function that they may serve in the formulation. Alternatively or additionally, certain pharmaceutically acceptable excipients may be chosen for their ability to facilitate the production of stable dosage forms, enhance bioavailability, and/or minimize side effects.

Excipients that may be used include buffering agents, carriers, diluents, fillers, binders. disintegrants, lubricants, glidants, granulating agents, coating agents, wetting agents, solvents, co-solvents, suspending agents, emulsifiers, coloring agents, anticaking agents, humectants, chelating agents, plasticizers, viscosity agents, antioxidants, preservatives, stabilizers, and surfactants. The skilled artisan will appreciate that certain pharmaceutically acceptable excipients may serve more than one function and may serve alternative functions depending on how much of the excipient is present in the formulation and what other ingredients are present in the formulation.

In embodiments in which the formulation includes an antibody or antigen binding fragment thereof. the formulation may include about 1 mg/ml to about 150 mg/ml of the antibody or antigen-binding fragment thereof. The formulation may also include at least one excipient as described above. An example of a suitable excipient is a buffering agent such as histidine, acetate, citrate, or phosphate. In one embodiment, the formulation comprises about 5 mM to about 100 mM buffering agent. The formulation may be isotonic.

The therapeutically effective concentration or dosage of active ingredient administered to a subject may vary depending on, for example, the nature of the formulation, mode of administration, particular condition to be treated, and condition and mass of the patient. Dosage levels are typically sufficient to achieve a tissue concentration at the site of action that is at least comparable to a concentration that has been shown to be active in vitro, in vivo, ex vivo, or in tissue culture. In an example, an anti-β1 integrin antibody is provided at a concentration of 0.1 mg/ml. In an example, an anti-CD93 antibody is provided at a concentration of 0.1 μg/μl.

### Diagnostic Methods

The methods described herein are suitable for diagnosing or predicting relapse of INS and/or predicting progression to end stage kidney disease. Levels of CD93 protein in urine are elevated in patients with INS compared to subjects without nephrotic disease. See Examples 2 and 7. Higher levels of urinary CD93, compared to healthy subjects, indicate a higher likelihood of kidney disease progression compared to lower levels of urinary CD93. See Example 8. In some embodiments, higher levels of urinary CD93 predict a higher likelihood of kidney disease progression compared to lower levels of urinary CD93 for patients with FSGS. Higher levels of urinary CD93, compared to healthy subjects, indicate a higher likelihood of developing proteinuria compared to lower levels of urinary CD93. See Example 8. Lower levels of urinary CD93 (i.e., levels closer to those of healthy subjects), indicate a shorter time to complete remission compared to higher levels of urinary CD93. See Example 8.

A high level of CD93 in urine may indicate a disease state or likelihood of onset of, or increasing severity of, a disease state as described above. The level of CD93 in urine may be considered high, or higher than normal or healthy subjects, when it is about 750 ng/g creatinine or greater, such as about 1,000 ng/g creatinine to about 10,000 ng/g creatinine, about 2,000 ng/g creatinine to about 10,000 ng/g creatinine, about 3,000 ng/g creatinine to about 10,000 ng/g creatinine, about 4,000 ng/g creatinine to about 10,000 ng/g creatinine, about 5,000 ng/g creatinine to about 10,000 ng/g creatinine, about 6,000 ng/g creatinine to about 10,000 ng/g creatinine, about 7,000 ng/g creatinine to about 10,000 ng/g creatinine, about 750 ng/g creatinine to about 9,000 ng/g creatinine, about 750 ng/g creatinine to about 8,000 ng/g creatinine, about 750 ng/g creatinine to about 7,000 ng/g creatinine. about 750 ng/g creatinine to about 6,000 ng/g creatinine, about 750 ng/g creatinine to about 5,000 ng/g creatinine, or about 750 ng/g creatinine to about 4,000 ng/g creatinine.

The level of CD93 in urine may be considered low, or normal or healthy, when it is less than about 750 ng/g creatinine. In some healthy subjects, CD93 may be present at up to about 1000 ng/g creatinine. Normal CD93 levels in urine may be about 0 ng/g creatinine to about 750 ng/g creatinine, such as about 50 ng/g creatinine to about 750 ng/g creatinine. about 150 ng/g creatinine to about 750 ng/g creatinine, about 250 ng/g creatinine to about 750 ng/g creatinine, about 350 ng/g creatinine to about 750 ng/g creatinine, about 450 ng/g creatinine to about 750 ng/g creatinine, about 550 ng/g creatinine to about 750 ng/g creatinine, about 0 ng/g creatinine to about 650 ng/g creatinine, about 0 ng/g creatinine to about 550 ng/g creatinine, about 0 ng/g creatinine to about 450 ng/g creatinine. about 0 ng/g creatinine to about 350 ng/g creatinine. about 0 ng/g creatinine to about 250 ng/g creatinine, or about 0 ng/g creatinine to about 150 ng/g creatinine. A level of 0 ng/g creatinine may indicate no CD93 present in urine, or no detectable amount of CD93.

CD93 and creatinine levels may be measured by any method known in the art, such as an antibody detection method In some implementations. CD93 levels are not normalized to creatinine levels.

A nephrologist or other medical professional may utilize results of a diagnostic method disclosed herein to implement a therapeutic method described herein and/or administer a pharmaceutical composition described herein.

### EXAMPLES

The following examples illustrate various aspects of the disclosure, and should not be considered limiting.

### Example 1: CD93 Expression in GEnC

De-identified paraffin-embedded human kidney samples from children and adults with active, biopsy-proven MCD (n=27) or FSGS (n=7), and 9 adult subjects without history of glomerular disease (controls) were obtained from the Histology Subspecialties Laboratory at the University of Colorado and from the Hospital Vall d′Hebron (HVdH) (Spain). Slides were deparaffinized and rehydrated according to standard protocols. Antigen retrieval was then performed leaving slides in citrate buffer at 96°° C. for 45 minutes. After cooling, slides were washed for 10 minutes in 0.1M glycine/TBST (Tris-buffered saline with Tween), and this was followed by permeabilization with 0.1% Triton X-100 for 10 minutes. Then, slides were placed in 10 mg/mL cold sodium borohydride/HBSS solution for 40 minutes. After that, slides were blocked with a solution 1:1 of superblock and 5% bovine serum albumin (BSA) in TBST for 1 hour. This was followed by overnight incubation with rabbit anti-CD93 (Sigma-Aldrich, catalog #HPA009300, dilution 1:100) and with the rhodamine lectin ulex europaeus agglutinin I (Vector, RL-1062-2, dilution 1:1000) as endothelial-specific marker. For the negative control, the primary antibody was omitted. After washing slides, goat anti-rabbit secondary antibody (dilution 1:400) was added to the slides, and these remained at room temperature for 2 hours. Finally, slides were washed and mounted with Prolong Gold antifade mounting medium (Invitrogen). Images were captured using Keyence BZ-X810 fluorescence microscope and analyzed with Image J.

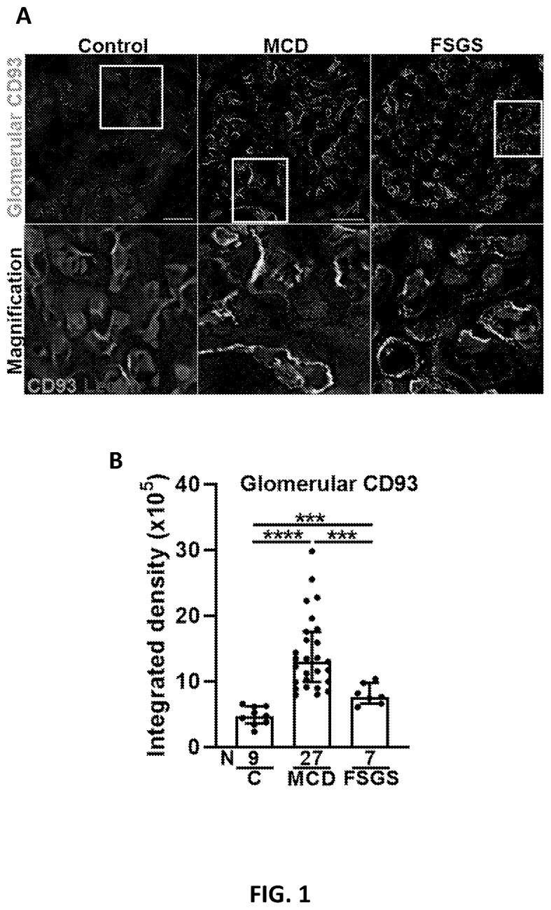

Results are shown in FIGS. 1A and 1B. In FIG. 1A, representative images (control=1, MCD=1, FSGS=1) were captured at 40× and scale bars=100 μm. Immunofluorescence shows that CD93 colocalized with the endothelial-specific lectin ulex europaeus agglutinin I.

Results show, in FIG. 1B, that CD93 expression in glomeruli was significantly higher in patients with MCD compared to controls (“C”) and in patients with FSGS compared to controls (N=number of subjects; mean±SD: (***p≤0.001; ****p≤0.0001). Results in FIG. 1B also show that CD93 expression in glomeruli was significantly higher in patients with MCD compared to patients with FSGS (***p≤0.001). The results demonstrate that CD93 is overexpressed in the glomerular endothelium of patients with MCD or FSGS.

### Example 2: CD93 Levels in Urine and Serum

Urine and serum samples were obtained from children with MCD/INS in relapse and from controls. Human biosamples were processed using standard protocols and stored at −80° C. until used for testing. Quantification of CD93 in blood (dilution 1:50) and urine (undiluted) was performed using a commercial CD93 ELISA Kit (R&D #DCD930) according to the manufacturer's protocol. Urine CD93 measurements were normalized to urine creatinine concentration.

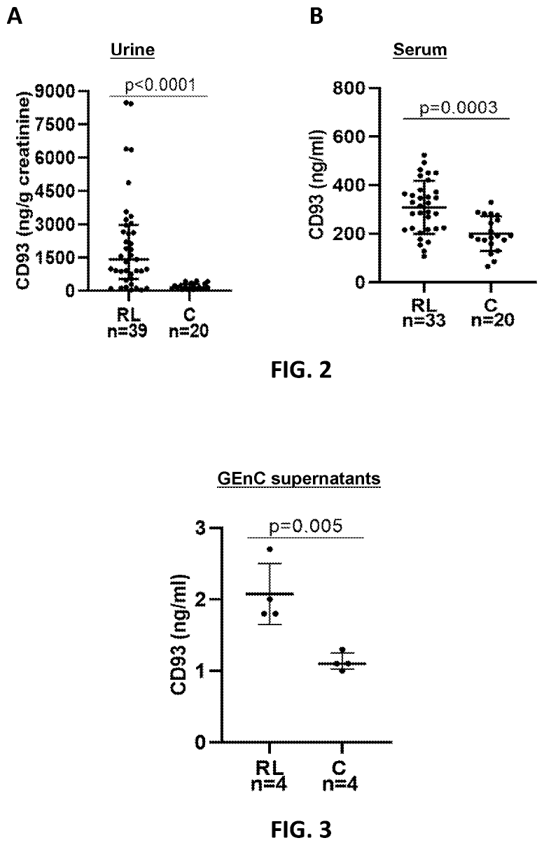

Results are shown in FIGS. 2A-2B. In urine (FIG. 2A), the median CD93 level in MCD/INS (“RL”; n=39) was nearly ten times higher than in controls (“C”; n=20) (median±interquartile range: p<0.0001), and 31 of 39 (79.4%) children with MCD/INS demonstrated urinary CD93 levels above the 95^{th} percentile for controls.

In serum (FIG. 2B), CD93 was higher in MCD/INS (“RL”; n=33) than in controls (“C”; n=20) (mean +SD: 60% increase over the control mean: p=0.0003), and 14 of 33 (42.4%) children with MCD/INS demonstrated levels greater than 2 standard deviations above the mean for controls. The results demonstrate that CD93 is elevated in urine and blood in MCD and INS. No correlation was found between urine and serum CD93 levels (data not shown).

### Example 3: GEnC Shedding of CD93

Human GEnC were cultured in 6-well plates. When fully differentiated, 10% unpooled serum samples from 6 patients with nephrotic syndrome (including MCD and SSNS (steroid sensitive nephrotic syndrome)) during relapse or 6 healthy subjects was added in FBS-free media. After 24 hours, cells were washed 5 times with phosphate buffered saline (PBS) to remove any remaining human sera from the wells. This was followed by the addition of fresh media. without human sera, to the 6-well plates for another 24 hours. Next. GEnC supernatants were collected (undiluted) to measure shed CD93 by commercial ELISA, as described above in Example 2.

Results are shown in FIG. 3. Supernatants obtained from human glomerular endothelial cells previously exposed to disease sera demonstrated significantly (**p≤0.01) higher CD93 levels than those exposed to healthy sera. The results demonstrate that that GEnC are a source of shed CD93 in response to INS sera.

### Example 4: Podocyte Activation by CD93

A human podocyte cell line was cultured in 6-well plates coated with fibronectin (1 mg/ml diluted in PBS). Upon full differentiation, recombinant CD93 (Sino Biological, cat. #12589-H08H, aa1-580) at a concentration of 0.1 μg/ml was added to culture media for several timepoints (0, 0.5, 1, 3, 6, and 24 hours, n=2). In a separate set of experiments, a β1 integrin inhibitor (monoclonal antibody 13, mAb 13) was mixed with culture media at 0.1 mg/ml and added to the fully differentiated human podocytes 20 minutes prior to adding recombinant CD93 at the foregoing timepoints (n=2 each). Next, plated podocytes were scraped, and proteins were collected using a mixture of M-PER (mammalian protein extraction reagent from Thermo Scientific), protease inhibitor (Roche). and phosphatase inhibitor (Roche). Protein quantification was performed using a Bradford assay (Bio-Rad, Hercules, CA). Sample proteins were separated on 4-12% SDS polyacrylamide gels and transferred to Immobilon-FL PVDF membranes (Merck Millipore Ltd). The membranes were blocked using Super Block (Thermo Scientific) for 1 hour at room temperature. Next membranes were incubated overnight at 4° C. with monoclonal rabbit anti-phospho-FAK (Tyr397) (Thermo Fisher, cat. #700255, dilution 1:5000) or monoclonal rabbit anti-GAPDH (Cell signaling, cat. #2118, dilution 1:5000). Membranes were washed and subsequently incubated with goat anti rabbit-HRP (Cell Signaling #7074, 1:6000) as a secondary antibody.

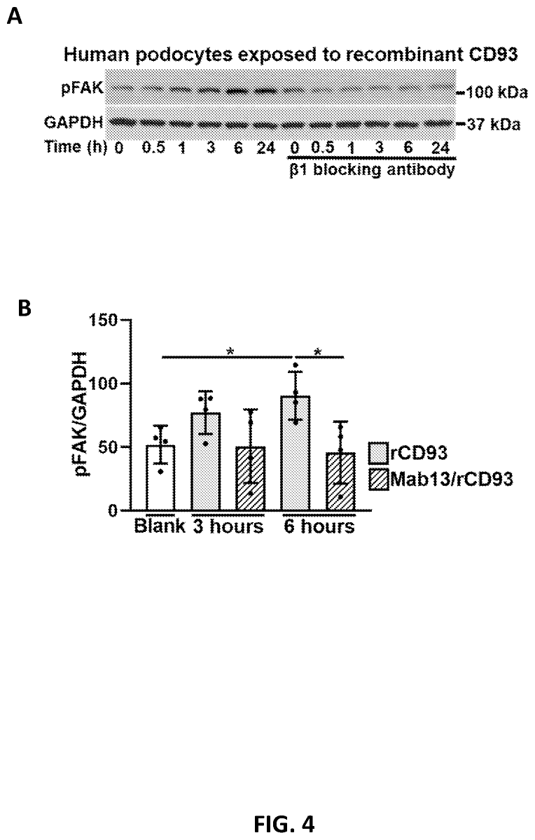

Western blot results are shown in FIG. 4A and densitometry analysis (arbitrary units), including 3 time points (0, 3 and 6 hours) from 4 independent experiments is shown in FIG. 4B. Immortalized human podocytes cultured with recombinant CD93 led to an increase in pFAK expression by 6 hours (FIG. 4B, time 0 (“Blank”) vs 6 hours, *p≤0.05). Podocyte FAK activation was time-dependent. pFAK expression was abrogated when podocytes were preemptively cultured with the β1 integrin inhibitor mAb 13 (FIG. 4B, rCD93 at 6 hours vs Mab13/rCD93 at 6 hours, *p≤0.05).

### Example 5: Mediation of Podocyte Activation by CD93 Shed in MCD Sera

Human podocytes were cultured as described for Example 4, except 10% sera from children with MCD/INS relapse or control subjects was added for 24 hours Cells were also cultured with and without the addition of an antibody that recognizes the extracellular domain of CD93 (Sigma #9300, 0.1 μg/μl, amino acid sequence 481-581). Proteins from cell lysates were collected and Western blots were performed as in Example 4.

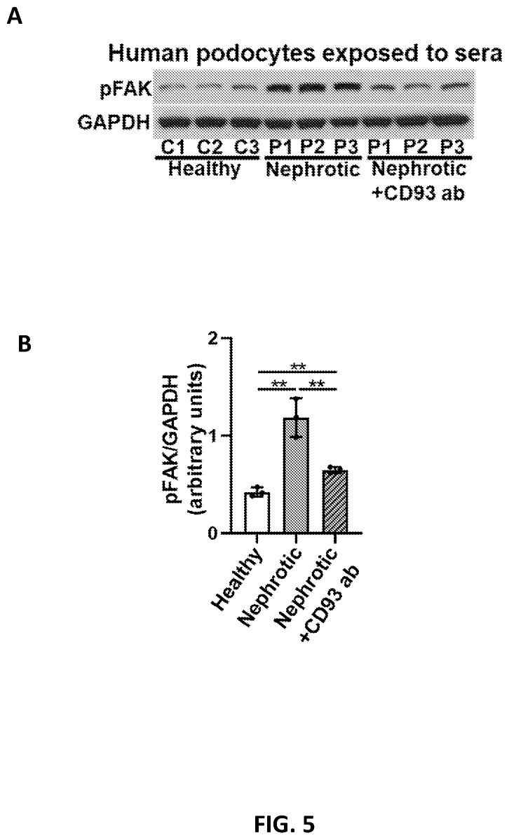

Results are shown in FIGS. 5A (Western blot) and 5B (densitometry). The results show an increase in FAK activation in podocytes exposed to INS in relapse sera (“Nephrotic”) compared to controls, but this was mitigated when podocytes were exposed to the same INS sera mixed with an antibody raised against the extracellular domain of CD93 (“Nepthrotic+CD93 ab”) (FIG. 5B. n=3; mean±SD: **p<0.01). The results demonstrate that CD93 shed into serum causes podocyte injury, which can be prevented by blocking CD93.

### Example 6: Transcriptome Analysis

Transcriptomic data from microdissected human glomeruli from children and adults with MCD or FSGS and controls was analyzed.

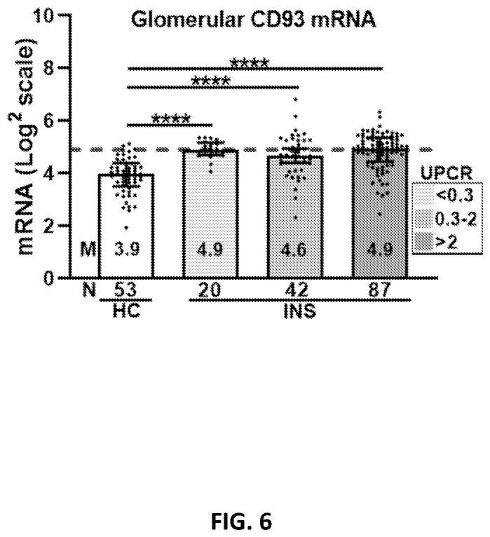

Results are presented in FIG. 6, in which data is presented as median±interquartile range, the dashed line represents the 95^{th} percentile cut-off value for the control group (“HC”), INS=MCD and FSGS samples collectively, M=median, N number of participants, UPCR=urine protein-to-creatinine ratio, and ****p≤0.0001. The INS bars from left to right show UPCR <0.3. 0.3-2. and >2. Results show that CD93 mRNA expression is higher in patients. regardless of the underlying histological pattern or degree of proteinuria (as represented by UPCR), than in controls.

### Example 7: CD93 Levels in Urine and Serum

Urine and serum samples were obtained from 228 children and adults with any form of INS (i.e., MCD, FSGS. steroid sensitive nephrotic syndrome (SSNS), steroid resistant nephrotic syndrome (SRNS), frequent relapsing nephrotic syndrome (FRNS), and infrequent relapsing nephrotic syndrome (IRNS)) during remission, active disease, and relapse, and from controls. Human biosamples were processed and CD93 was quantitated as described in Example 2.

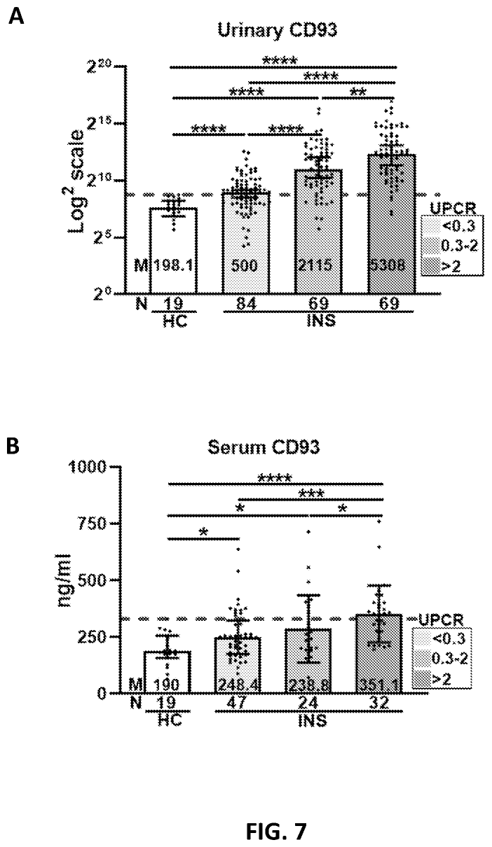

Results are shown in FIGS. 7A-7B, in which data is presented as median±interquartile range, the dashed line represents the 95^{th} percentile cut-off value for the control group (“HC”), M=median, N number of participants, UPCR =urine protein-to-creatinine ratio, *p≤0.05. **p≤0.01, ***p≤0.001. and ****p<0.0001. The INS bars from left to right show UPCR<0.3, 0.3-2. and >2. In urine (FIG. 7A), CD93 levels were significantly higher in INS subjects than in controls (p<0.0001 regardless of UPCR). CD93 levels increased as proteinuria increased. CD93 levels were above the normal limit in 89.1% of patients with active disease and remained high in 51.1% of patients in complete remission.

In serum (FIG. 7B), CD93 levels were significantly higher in INS subjects than in controls regardless of UPCR. CD93 levels increased as proteinuria increased. The results demonstrate that CD93 is elevated in urine and blood in INS. Between urine and blood, CD93 levels were markedly higher in the former, which suggests that CD93 is not simply filtered out through the leaky filtration barrier but instead is primarily shed from the kidney.

### Example 8: Clinical Outcome Associations

Urinary and serum CD93 levels were examined for their associations with clinical outcomes. Quartiles of CD93 were used to aid in data visualization. CD93 was analyzed to predict time to complete remission (i.e., a faster response to treatment) among those with active disease. time to relapse (i.e., risk of future proteinuria) among those in complete remission, and time to end-stage kidney disease (ESKD) or 40% loss in estimated glomerular filtration rate (eGFR) in all subjects. Kaplan-Meier curves were used for data visualization. Cox-Proportional hazards models (adjusting for diagnosis [FSGS vs. MCD], proteinuria [log transformed UPCR] at the time of CD93 collection, and histology [interstitial fibrosis]) were used for analyses of time to event.

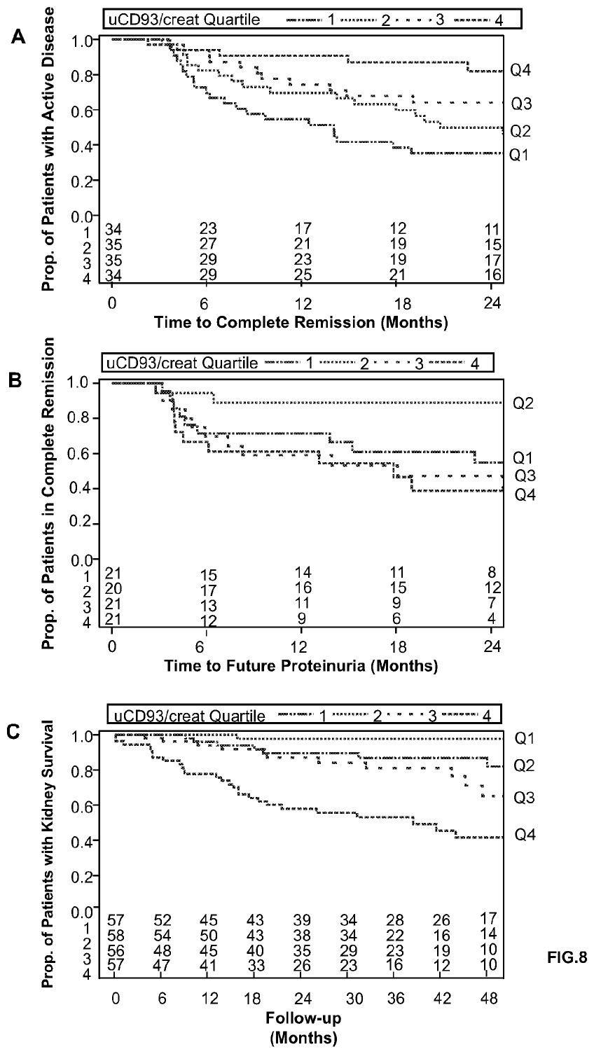

Results are shown in FIGS. 8A-8C. In patients with all forms of INS, a lower urinary CD93 was associated with shorter time to complete remission (FIG. 8A, hazard ratio (HR) 0.75, p<0.001): whereas a higher urinary CD93 was a predictor of kidney disease progression (FIG. 8C, HR 1.57, p<0.0001) and also showed a trend toward higher risk of future proteinuria (FIG. 8B, HR 1.35, p=0.06). When patients were analyzed according to the underlying histological pattern (FSGS vs MCD), urinary CD93 associated with time to complete remission in both groups and to kidney disease progression only in patients with FSGS (data not shown). Each of serum CD93 and glomerular mRNA expression failed to predict the studied clinical outcomes (data not shown).

### Example 9: Effect of Sera on CD93 in Human Glomerular Endothelial Cell Lysates

Human GEnC were cultured until fully differentiated. Then, 10% unpooled serum samples from 7 children with idiopathic nephrotic syndrome in relapse or 6 healthy children was added. Twenty-four hours later, cells were scraped, and proteins collected as described above for Example 4. Samples were subjected to Western blotting for CD93 as described in Example 4.

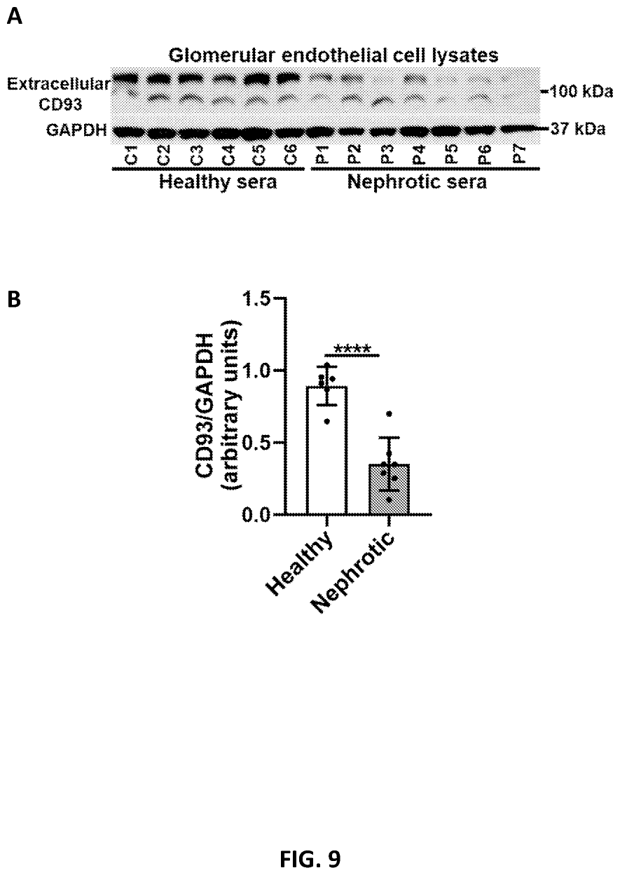

Results are presented in FIGS. 9A and 9B. Results show that CD93 expression in human glomerular endothelial cell lysates following incubation with sera from patients with INS during relapse (“P”) was significantly (****p≤0.0001) reduced compared to healthy subjects (“C”).

### Example 10: Co-Immunoprecipitation Studies

Co-immunoprecipitation studies were performed to identify interactions between soluble CD93 and podocyte β1 integrin. Immortalized podocytes were incubated with recombinant CD93 (Sino Biological, cat #12589-H08H, 0.1 μg/ml) or serum from a patient with INS in relapse for 1 and 3 hours. After incubation, cells were placed on ice and lysed with 1× RIPA buffer (Cell signaling Cat. #9806). After quantification. 150 μg of protein lysate was precleared with 20 μl agarose beads protein A/G Plus (Santa Cruz Biotechnology sc-2003). The precleared supernatant was incubated overnight at 4° C. with rotation with 2 μg of anti-CD93 antibody (Sigma-Aldrich, Cat. #HPA009300). The next day, the immunocomplex was pulled down by incubation with 20 μl agarose beads protein A/G slurry following a series of centrifugation/washes with ice-cold PBS and eluted by boiling the samples in 1×western blot loading buffer. The same approach, but omitting the CD93 antibody. was used as a negative control.

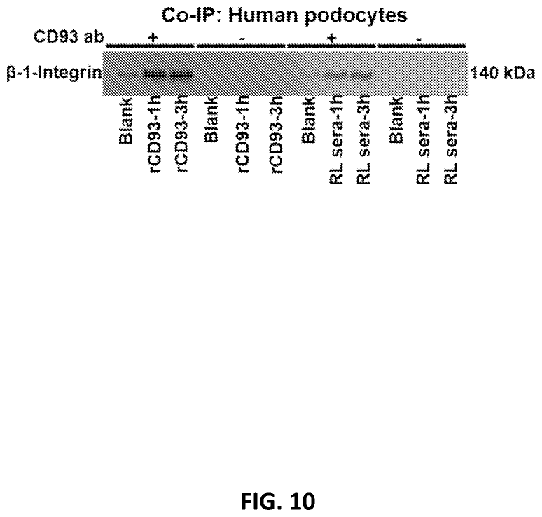

Results are presented in FIG. 10 and show that CD93 binds to β1 integrin in cultured human podocytes.

### Example 11: Investigation of CD93 in a Rat Model of Nephrotic Syndrome

The presence of CD93 in glomeruli and urine in the puromycin aminoglycoside (PAN) rat model of nephrotic syndrome was investigated. In brief, male Wistar rats (body weight ˜150 g, age ˜45-50 days) received a single tail vein injection of puromycin aminoglycoside (50 mg/kg: 5 groups) or saline (4 groups) on Day 0. Serial urine samples were collected for quantification of urine protein creatinine ratio (UPCR) and urinary CD93 (Novus, cat. #NBP2-70033, dilution 1:2). All procedures were approved by the Institutional Animal Care and Use Committee, in accordance with the NIH Guide for the Care and Use of Laboratory Animals.

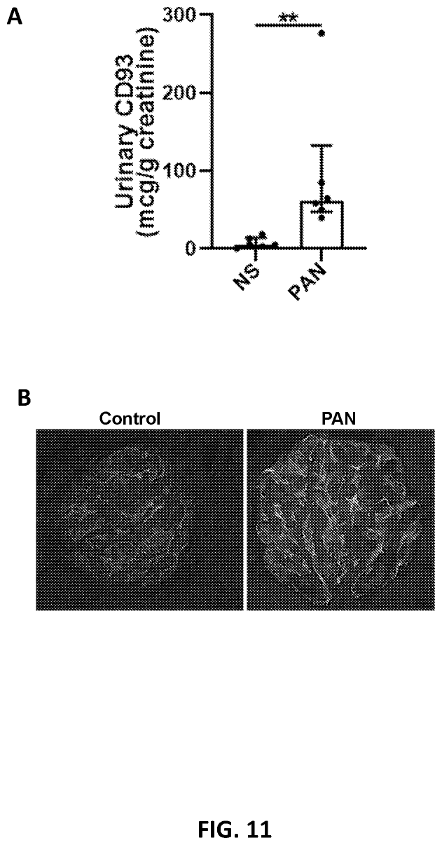

Results are presented in FIGS. 11A and 11B. CD93 levels in the urine of PAN rats were significantly (**p<0.01) higher than in normal saline controls (“NS”) (FIG. 11A). CD93 expression was higher in PAN rats compared to controls (FIG. 11B).

Although various embodiments have been described above with a certain degree of particularity, or with reference to one or more individual embodiments, those skilled in the art could make numerous alterations to the disclosed embodiments without departing from the spirit or scope of this disclosure. Other embodiments are therefore contemplated. All matter contained in the above description and the accompanying drawings is illustrative only of particular embodiments and not limiting. Changes in detail, structure, or order of operation of steps of a method may be made without departing from the basic elements described herein.

1. A method of treating idiopathic nephrotic syndrome in a subject, the method comprising reducing urinary and/or blood levels of CD93.

2. The method of claim 1, wherein the idiopathic nephrotic syndrome is selected from minimal change disease, focal segmental glomerulosclerosis, steroid sensitive nephrotic syndrome, steroid resistant nephrotic syndrome, frequent relapsing nephrotic syndrome, and infrequent relapsing nephrotic syndrome.

3. The method of claim 1, wherein urinary and/or blood levels of CD93 are reduced by administering at least one anti-CD93 antibody.

4. The method of claim 1, wherein urinary and/or blood levels of CD93 are reduced by reducing or inhibiting CD93 release from glomerular endothelial cells.

5. The method of claim 1, wherein urinary and/or blood levels of CD93 are reduced by administering a composition capable of reducing or inhibiting CD93 release from glomerular endothelial cells.

6. The method of claim 1, further comprising preventing or reducing podocyte activation, and/or reducing proteinuria.

7. (canceled)

8. (canceled)

9. The method of claim 6, wherein podocyte activation includes phosphorylation of focal adhesion kinase and reduction of activation and/or proteinuria includes administration of at least one anti-CD93 antibody.

10. A method of preventing or reducing podocyte injury and/or proteinuria in a subject, the method comprising reducing or inhibiting CD93-mediated podocyte activation.

11. (canceled)

12. (canceled)

13. The method of claim 10, wherein podocyte activation is reduced by administering at least one anti-CD93 antibody.

14. The method of claim 10, wherein podocyte activation is reduced by reducing or inhibiting CD93 release from glomerular endothelial cells.

15. The method of claim 10, wherein podocyte activation is reduced by administering a composition capable of reducing or inhibiting CD93 release from glomerular endothelial cells.

16. A method of treating an idiopathic nephrotic syndrome in a subject, the method comprising reducing or inhibiting CD93 protein release from glomerular endothelial cells.

17. The method of claim 16, wherein the idiopathic nephrotic syndrome is selected from minimal change disease, focal segmental glomerulosclerosis, steroid sensitive nephrotic syndrome, steroid resistant nephrotic syndrome, frequent relapsing nephrotic syndrome, and infrequent relapsing nephrotic syndrome.

18. The method of claim 16, wherein the method includes administering a composition capable of reducing or inhibiting CD93 release.

19. The method of claim 18, wherein CD93 levels are reduced in urine and/or blood by the administering step.

20. The method of claim 18, wherein podocyte activation and/or proteinuria is reduced by the administering step.

21. A method of treating proteinuria in a subject with elevated serum and/or urine CD93 levels, the method comprising administering a composition capable of reducing CD93 levels or inhibiting CD93 release.

22. The method of claim 21, wherein elevated CD93 comprises a CD93 level of 750 ng or greater per gram of creatinine.

23. The method of claim 22, wherein the CD93 level is a urinary level.

24. The method of claim 21, wherein administering reduces podocyte activation and/or proteinuria.

25. The method of claim 21, wherein the patient has a urinary protein/creatinine ratio (UPCR) of greater than 0.3 g/g.

26. The method of claim 10, wherein administering step reduces urinary and/or blood CD93 levels in the subject.
